(19) United States
(12) Patent Application Publication
(10) Pub. No.: US 2018/0185568 A1
(43) Pub. Date: Jul. 5, 2018

(54) REUSABLE BOTTLE ATTACHMENT

(71) Applicants: Greg M. Griffin, Los Angeles, CA (US); Jacob Semans, Los Angeles, CA (US)

(72) Inventors: Greg M. Griffin, Los Angeles, CA (US); Jacob Semans, Los Angeles, CA (US)

(21) Appl. No.: 15/395,740

(22) Filed: Dec. 30, 2016

(51) Int. Cl. A61M 3/02; B65D 35/36

(52) U.S. Cl. CPC A61M 3/0279; A61M 2202/04; A61M 3/0262; B65D 35/36

(57) ABSTRACT

It is an object of the present invention to provide an attachment mechanism capable of being attached to a variety of solution sources. The attachment mechanism is uniquely configured to provide at least two different seals with a solution source. The attachment mechanism may also be used in a variety of applications.

### CROSS REFERENCE RELATED APPLICATIONS

This application claims priority to co-filed Design Application entitled “Enema Device” filed on Dec. 30, 2016 the contents of which are incorporated herein by reference in its entirety.

### TECHNICAL FIELD

The present invention is related to a mechanism that is capable of being attached to a plurality of container openings for a variety of functions.

### BACKGROUND

Several bottle attachments are available on the market which may be used to discharge fluid out of, or contain fluid within, a squeeze bottle. Such attachments include straws or syringes attached to over-the-counter douche, enema, and related fluid-dispensing products. Many of these attachments, however, are not durable, comfortable, easy to clean, or readily usable more than once.

In addition, such bottle attachments are designed and manufactured for use only with certain bottles, such as in douche and enema systems that include a straw attachment that is screwed onto the cap of a bottle. These attachments cannot be easily, safely, or securely used with other bottles that have varying girths, lengths, or diameters. If used with other bottles, such attachments may not fit securely onto those bottles, which thus results in leakage as well as difficulty of use.

A bottle attachment is needed that is durable, comfortable, portable, easy to clean, and readily usable more than once, and that can be used easily, safely, and securely with a variety of bottles, including bottles with varying girths, lengths, and diameters.

### BRIEF SUMMARY

Described herein is a portable device that is attachable to a plurality of solution sources. In one embodiment the portable device comprises an attachment device that includes at least an inner portion and an outer portion. The inner portion comprises a fluid path for distributing a solution from the solution source and the outer portion is configured to surround a neck finish of the solution source. The inner portion creates a first seal with the solution source, while the outer portion creates a second seal with the solution source.

In one embodiment, the outer portion of the attachment device sounds a neck support ring of the solution source. The outer portion may also comprise a first groove that is configured to interlock with one or more grooves located in the neck support ring of the solution source. In addition, the outer portion may comprise a lip that is configured to attach under the neck support ring of the solution source.

In one embodiment, inner portion comprises one or more inside grooves. The inside grooves may be configured to be completely or partially inserted into the mouth of the solution source.

In one embodiment a portable reusable enema device is described that comprises a silicon tube with pyramid-shaped end and an attachment end attached together via an elongated portion. The portable reusable enema device is configured to provide a first seal with a solution source and a second seal with a user's rectum.

In one embodiment, the silicon tube of the portable reusable enema device is less than 5.5 inches in height.

In various embodiments, the solution inside the solution source used the portable reusable enema device may be one of water or a saline solution.

In one embodiment, the solution sourced used with the portable reusable enema device may be a water bottle containing filtered water.

In one embodiment, the portable reusable enema device further comprises a gripper portion that contains raised text. The gripper portion may be less than 1/16 inches in depth.

### BRIEF DESCRIPTION OF THE DRAWINGS

FIG. 1 is an exemplary embodiment of an attachment mechanism described herein;

FIG. 2 is an alternate view of the attachment mechanism of FIG. 1;

FIG. 3 is an alternate view of the attachment mechanism of FIG. 1;

FIG. 4 is an exemplary embodiment of an enema device described herein;

FIG. 5 is an alternate view of the enema device of FIG. 4;

FIG. 6 is an alternate view of the enema device of FIG. 4;

FIG. 7 is an exemplary bottle that is capable of being used with various embodiments described herein;

FIG. 8 illustrates an exemplary dimensional break down of the enema device of FIG. 4.

### DETAILED DESCRIPTION

The present invention is generally relates to an attachment mechanism capable of being utilized in various different applications. In one embodiment, the attachment mechanism is configured to be used as a bottle attachment that creates a secure seal that prevents leakage of fluids contained in the bottle.

### Attachment Mechanism

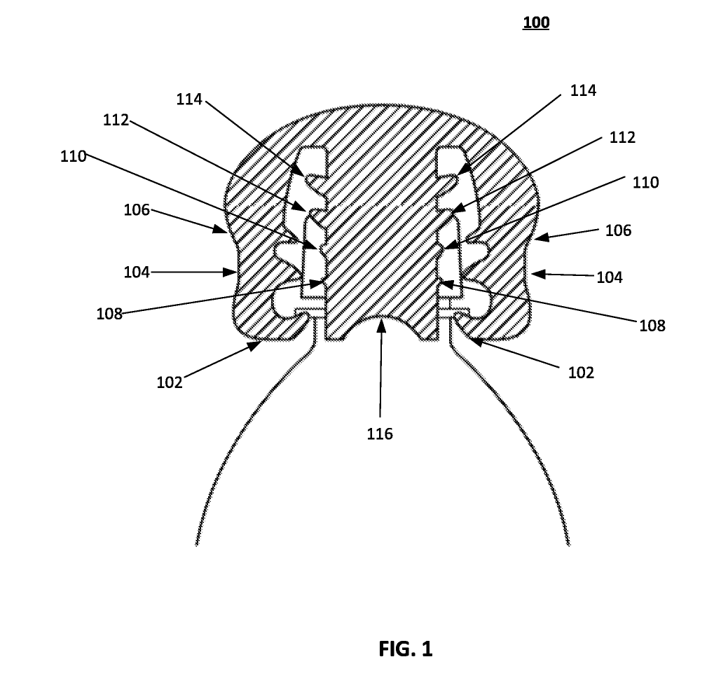

FIG. 1 illustrates an exemplary embodiment of attachment mechanism 100 from an internal view. Attachment mechanism 100 comprises of an inside portion (items 108-116) and an outside portion (items 102-106). The inside portion of attachment mechanism 100 is configured to provide a plugging seal with the mouth of a bottle. The outside portion of attachment mechanism 100 is configured to provide an overlap seal with the mouth of a bottle. The plugging seal and the overlap seal separately and in combination prevent fluid leakage from the bottle. In some embodiments attachment mechanism 100 may be made of a food grade silicone with a 40-70 grade of firmness. The grade of firmness may depend upon the application. In addition, as describe herein, attachment mechanism 100 may be utilized in a plurality of different applications.

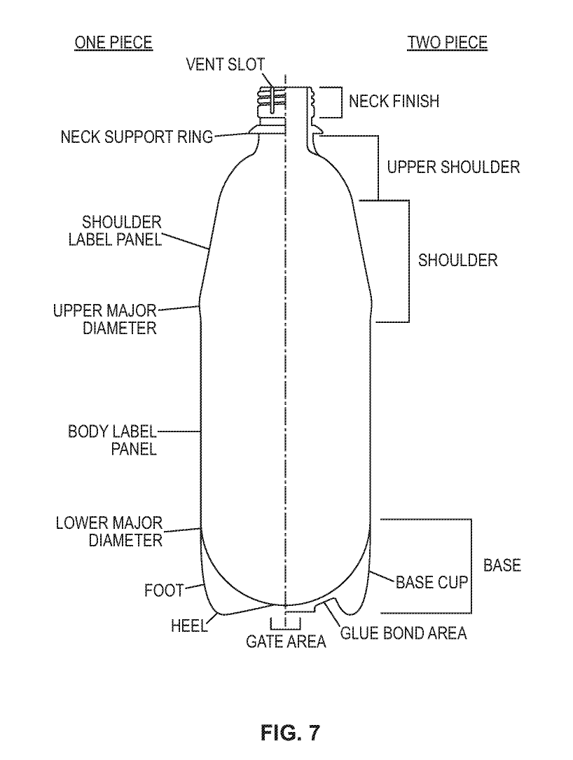

Outside portion of attachment mechanism 100 comprises of lip 102, lower groove 104, and upper groove 106. The outside portion may consist of all of or a portion of items 102-16. For example, in one embodiment, only lip 102 may be provided without lower groove 104 and upper groove 106. Lip 102 is configured to overlap the neck support ring on a bottle. FIG. 7 shows in exemplary embodiment of a bottle that may be used in connection with attachment mechanism 100. The bottle in FIG. 7 may be a variety of bottles including a plastic water bottle. Depending on the country of origin and the size and brand of the water bottle, the neck and collar of a commercially available water bottle can vary drastically. Attachment mechanism 100 is capable of attaching to a wide variety of water bottles, in part because of the silicone material and the configuration of the attachment mechanism. Lip 102 extends over and wraps under the neck support ring of a bottle in order to provide a secure connection to the bottle. When lip 102 is wrapped under the neck support ring it prevents attachment mechanism 100 from being able to be removed by pulling the attachment mechanism 100. In one embodiment, attachment mechanism is made of a silicone that has a degree of firmness between 50-70 degrees. A firmness in this range provides an optimal seal with the largest variety of water bottle neck finishes. In order to un-secure the connection between lip 102 and a bottle, lip 102 must be physically removed by interacting directly with lip 102. For example, a person may un-secure the connection by using his or her thumb to pry lip 102 away from the bottle.

Lower groove 104 attaches to one or more grooves located in the neck finish region of a bottle. Lower groove 104 provides stability for the attachment mechanism 100 by fitting in between (e.g. interlocking) one or more grooves in the neck finish region. In a typical bottle, the neck finishing region comprise of a plurality of outward protruding grooves that correspond to a plurality of groves on the inside of a corresponding bottle cap. The corresponding grooves inside the bottle cap and in the neck finish region of the bottle create a secure seal on the bottle mouth when the cap is properly installed on the bottle's mouth (e.g. by screwing on the bottle cap). Similarly, lower groove 104 is adapted to fit between one or more outward grooves in the neck finish to create a secure seal between the bottle and attachment mechanism 100. Lower groove 104 also provides stability for the attachment mechanism 100 such that the attachment mechanism 100 remains in a relative stable position while in use (e.g. as bottle top, enema device, and the like).

Upper groove 106 attaches to one or more grooves located in the neck finish region of a bottle, and is displaced above lower groove 104. Upper grove 106 provides added stability for the attachment mechanism 100 by fitting in between one or more grooves in the neck finish region. In one embodiment, upper groove 106 may be placed above the top groove in the neck finish region of a bottle. Similar to the lower groove 104, upper groove 106 provides stability for the attachment mechanism 100 such that the attachment mechanism 100 remains in a relative stable position while in use. In some embodiments, lower groove 104 and/or upper groove 106 may be omitted from the outside portion of attachment mechanism 100. The determination to exclude or include lower groove 104 and/or upper groove 106 may be based, at least in part, on the neck finish design of a bottle (e.g. groove design, groove number, etc.)

Inside portion of attachment mechanism 100 comprises of first inside groove 108, second inside groove 110, third inside groove 112, fourth inside groove 112, and plug 116. Plug 116 is designed to fit, at least partially, inside the mouth of a bottle. Plug 116 comprises a fluid path that is capable of facilitating the flow of a liquid from an attached bottle. In some embodiments, the inside portion of attachment 100 may only comprise one or more of the grooves. For example, inside portion may consist of only fourth inside groove 112 and plug 116. In another example, inside portion may consist of fourth inside groove 112, second inside groove 110, and plug 116. In one embodiment, inside grooves 108-112 are configured such that when the attachment mechanism 100 is correctly installed on a bottle that all of the grooves are physically inside the bottle's mouth. By having grooves 108-112 fully inserted into the mouth of a bottle a plugging seal is created. In some embodiments, it is possible to correctly install attachment mechanism 100 with a portion of plug 116 not fully inserted in the mouth of the bottle, so long as all of the inside grooves are fully inserted into the mouth of the bottle. In an alternative embodiment, depending on the length of the mouth of the bottle used with attachment mechanism 100 only one or more of grooves 108-112 may need to be inserted. The determination to exclude or include one or more inside grooves 108-112 may be based, at least in part, on the neck finish design of a bottle (e.g. groove design, groove number, etc.)

First inside groove 108 protrudes outward from plug 116 and extends around plug 116 (e.g. in a circular fashion). First inside groove 108 is smaller in area than second inside groove 110.

Second inside groove 110 protrudes outward from plug 116 and extends around plug 116 (e.g. in a circular fashion). Second inside groove 110 is smaller in area than third inside groove 112, but is larger in area than first inside groove 108. Second inside groove 110 is displaced above first inside groove 108, but below third inside groove 112.

Third inside groove 112 protrudes outward from plug 116 and extends around plug 116 (e.g. in a circular fashion). Third inside groove 112 is smaller in area than fourth inside groove 114, but is larger in area than second inside groove 110. Third inside groove 112 is displaced above second inside groove 110, but below fourth inside groove 114.

Fourth inside groove 114 protrudes outward from plug 116 and extends around plug 116 (e.g. in a circular fashion). Fourth inside groove 112 is larger in area than third inside groove 112. Fourth inside groove 114 is displaced above third inside groove 112.

The embodiment depicted in FIG. 1 illustrates four inside grooves. However, it is within the concept of the invention described herein to have any number of grooves extending from plug 116. Regardless of the number of inside grooves, they must increase in area as they are displaced closer to the top of attachment mechanism 100. This means inside grooves located at the bottom of plug 116 will be smaller (in terms of area) than inside grooves located higher up on plug 116.

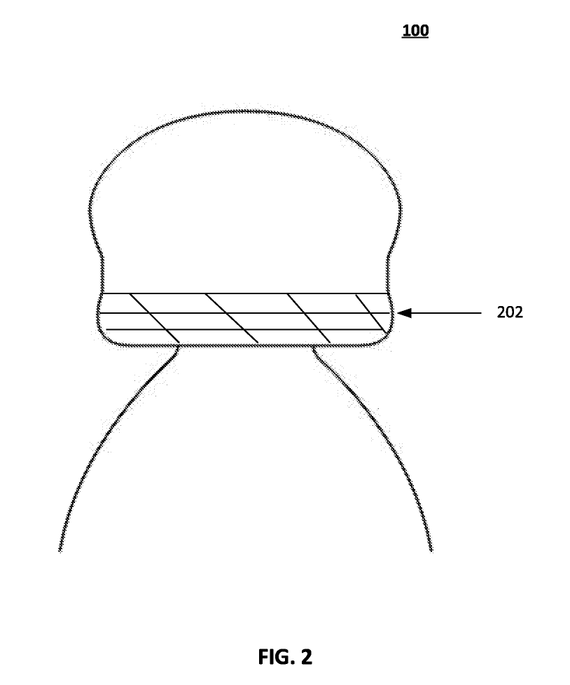

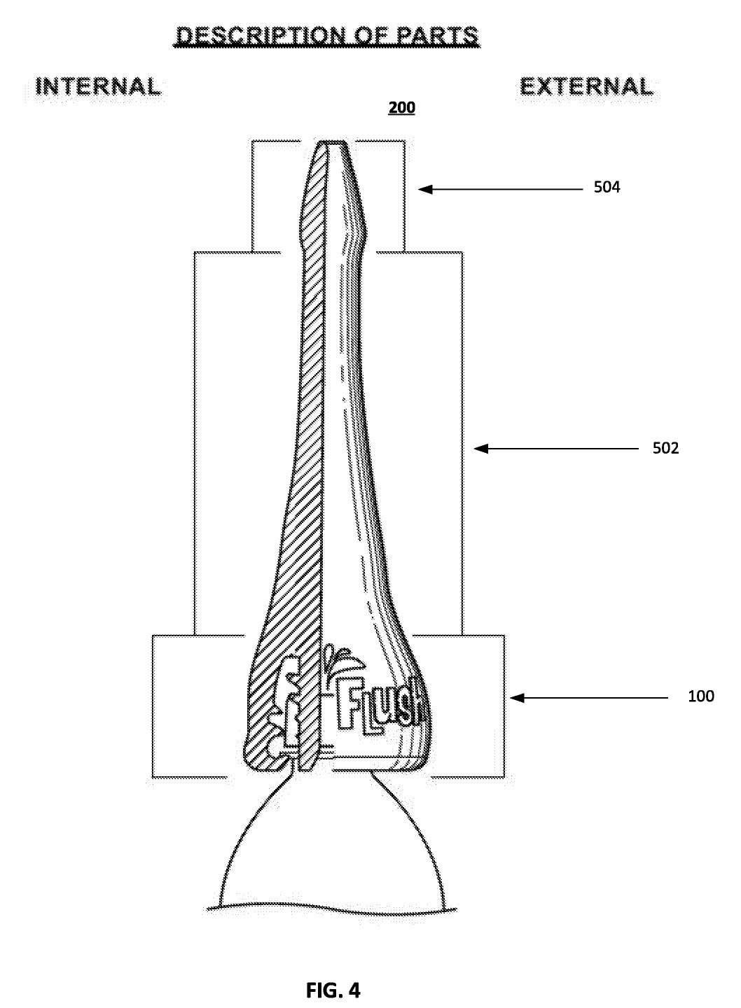

FIG. 2 illustrates an exemplary embodiment of attachment mechanism 100 from an outside view. Attachment mechanism 100 may comprise of gripper portion 202. Gripper portion 202 may be comprised of raised text (see “Flush” logo present in attachment portion 100 in FIG. 4) or ribbed text. Gripper portion 202 can be comprised of any material that provides a different and raised texture than the texture of the attachment mechanism 100. In one embodiment, the gripper portion 202 may protrude no more than 1/16 inches from the attachment mechanism 100. In some embodiments, the gripper portion 202 is made of the same material that the attachment mechanism 100 is made out of In other examples, gripper portion 202 may be made out of different material than attachment mechanism 100.

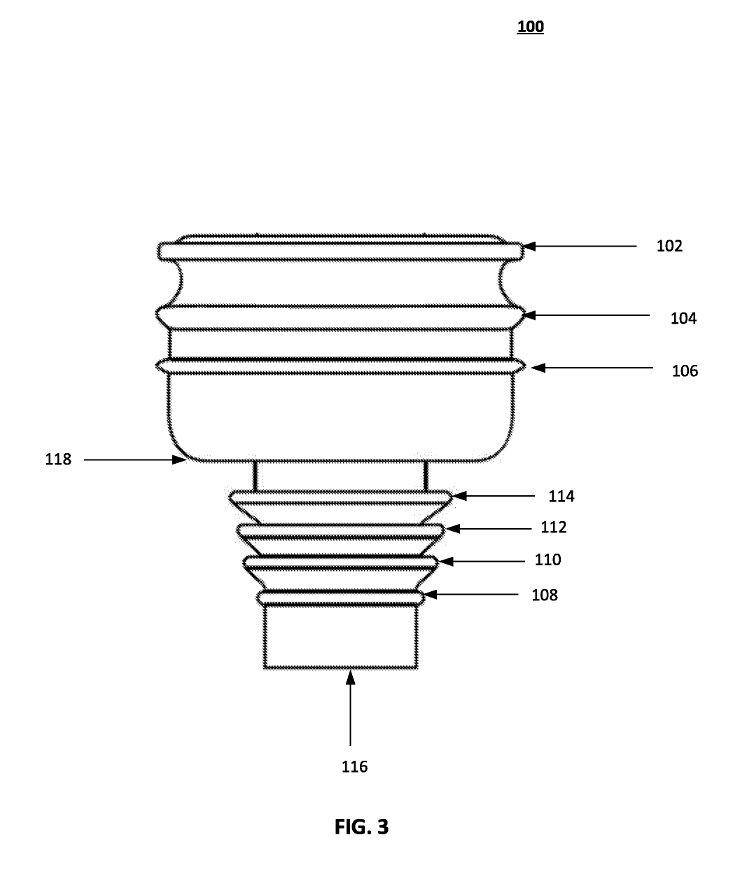

FIG. 3 illustrates an exemplary embodiment of attachment mechanism 100 from a view when the outside portion is inverted. Numbers in FIG. 3 correspond to like numbers in FIG. 1. From the angle displayed in FIG. 3 it is possible to see the top of the outside portion 118 of attachment mechanism 100. In some embodiments, plug 116 is inserted all the way into the mouth of a bottle such that the top of the mouth of the bottle contacts top of the outside portion 118. By inserting plug 116 to this depth it ensures that all inside grooves 108-114 are inside the bottle's mouth which provides for a plugging seal. Furthermore when installed correctly (as shown in FIG. 1) the top of the outside portion 118 may continue the fluid path of plug 116. This will allow for fluid to flow from a bottle to outside the top of attachment mechanism 100. In some embodiments, fluid from the attached bottle will flow through the fluid path of plug 116 and top of the outside portion 118 when the bottle is compressed in an inward direction (e.g. a squeeze). The compression creates the pressure needed to propel the fluid though the fluid path. In such an embodiment, attachment mechanism 100 may be used as an attachment on a sports drink to allow a user to transfer liquid from the sports drink to the user's mouth without the user's mouth contacting the attachment mechanism 100. In another embodiment, the top of the outside portion 118 may comprise a displaceable lid that is capable of being displaced in an open and a closed position. In the open position, the fluid in the fluid path may exit the attachment mechanism (e.g. by putting an inward pressure on the bottle, turning the bottle upside down, etc.) In the closed position, absent an intentional action (e.g. removing/tampering with attachment mechanism) the fluid in the fluid path will not exit the attachment mechanism.

### Enema Embodiment

FIG. 4 illustrates an exemplary embodiment of enema device 200, which is an embodiment of attachment mechanism 100 described in relation to FIGS. 1-3 above. Enema device 200 comprises of attachment mechanism 100, elongated portion 502, and tip 504. The configuration of attachment mechanism 100 has been described in relation to FIGS. 1-3. However, the fluid path created by plug 116 and the top of the outside portion 118 now extends through elongated portion 502 and tip 504, such that fluid is transferred from an attached bottle to outside of tip 504.

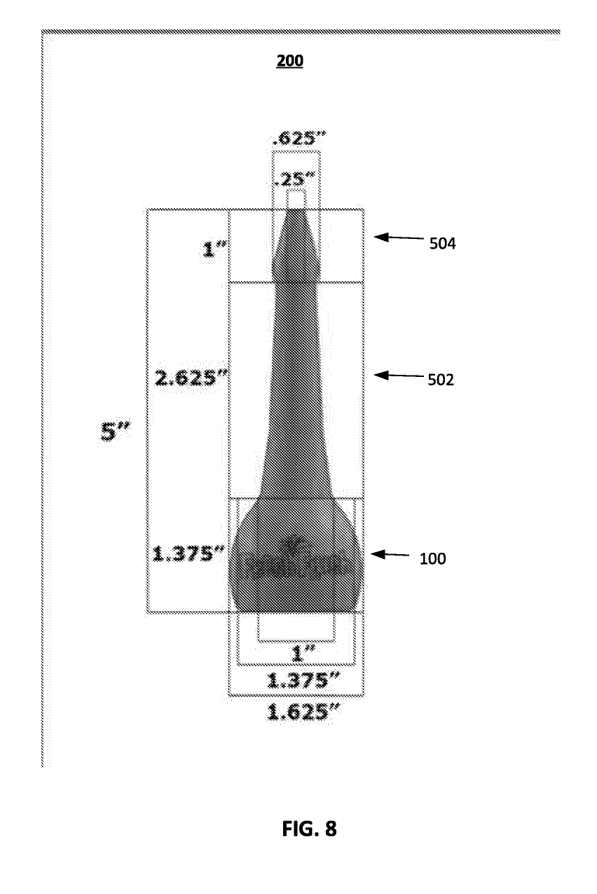

FIG. 8 illustrates an exemplary dimensional break down of enema device 200. Attachment mechanism 100 may be in the dimensions of 1.375 inches in height and 1.625 inches in width (at its widest point.) Elongated portion 502 may be in the dimensions of 2.625 inches in height and 1.375 inches in width (at its widest point.) Tip 504 may be in the dimensions of 1 inch in height and 0.625 inches in width (at its widest point.) An opening in tip 504 may be 0.25 inches wide. Enema device 200 may be comprised of silicone with a degree of firmness between 40-70 degrees.

Now with returned reference to FIG. 4, elongated portion 502 connects attachment mechanism 100 to tip 504 and comprises a fluid path for the transfer of fluids from an attached bottle (via the attachment mechanism 100) to tip 504. As depicted in FIG. 4 elongated portion 502 has a greater width at its base (i.e. closest to attachment mechanism 100) than at its top (i.e. closest to tip 504). This design enables, when the enema device 200 is use, elongated portion 502 to be at least partially inserted into a person's rectum. In one embedment, all of elongated portion 502 is inserted into a person's rectum and a seal is created between the person's rectum and the base of the elongated portion 502 and/or the attachment portion 100. Elongated portion 502 is uniquely designed for a comfortable insertion into a user's rectum. The length and girth of elongated portion 502 (specified below) are designed to work with most human body types and the wider base creates a seal against a user's rectum so water does not immediately flow back out during use.

Tip 504 is connected to elongated portion 502 and comprises a fluid path for the transfer of fluids from an attached bottle. Tip 504 further comprises an opening that allows an exit for the fluid path. As depicted in FIG. 4 tip 504 has a greater width and its base (i.e. closet to elongated portion 502) than at its top (i.e. furthest from elongated portion 502). The base of tip 504 also has a greater width than the top of the elongated portion 502 (i.e. the part of elongated portion 502 that connects to tip 504). Furthermore, the base of tip 504 is rounded (i.e. curved). The design of tip 504 enables the enema device 200 to have an increased level of comfort when inserted into a person's rectum. Furthermore, the design of tip 504 allows easy insertion of enema device 200 into a person's rectum due, at least in part, to the top of tip 504 being narrower than the curved base. Tip 504 is designed for ease and comfort of insertion into a variety of body types. The design is created with smooth, curved edges so as not to scrap the interior of the rectum like many other known enema devices.

Enema device 200 is configured to provide two seals. A first seal between a person's rectum and the enema device and a second seal between the enema device and a bottle. In some embodiments, the bottle may be a typical plastic water bottle. The water bottle may comprise of filtered water, a saline solution and the like. The first seal is created by inserting enema device 200 into a person's rectum such that the majority of the elongated portion 502 is inserted into the person's rectum. In some embodiments, the elongated portion 502 is fully inserted into the person's rectum such that the first seal is created between the top of attachment portion 100 and the said rectum. The first seal prevents leakage of fluid disbursed via the fluid path of enema device 200 during use. Without the first seal, when enema device 200 is in use, fluid disbursed via the fluid path would potentially leak uncomfortably and uncontrollably from the person's rectum. The first seal allows fluid distributed via the fluid path to remain sealed until the enema device 200 is removed from use (e.g. the elongated portion 502 is at least partially removed from the person's rectum.)

Enema device 200 is configured to provide a second seal between the enema device and a bottle. As described in relation to FIGS. 1-4, attachment mechanism 100 is configured to provide both a plugging seal and an overlapping seal with an attached bottle. The second seal (i.e. overlapping seal) and the first seal (i.e. plugging seal) created by the enema device 200 allows for a cleaner use of an enema device than those currently available.

Furthermore enema device 200 is configured to provide a third seal between the enema device and a person's rectum. In one embodiment, when in use, tip 504 and elongated portion 502 are fully inserted into the user's rectum, such that a seal is created between the user's rectum and the attachment portion 100. The seal prevents solution distributed via tip 504 from unintentionally leaking from the user's rectum. Current enema designs have a straw shaped insertion tube that causes water to leak from the user's rectum during use.

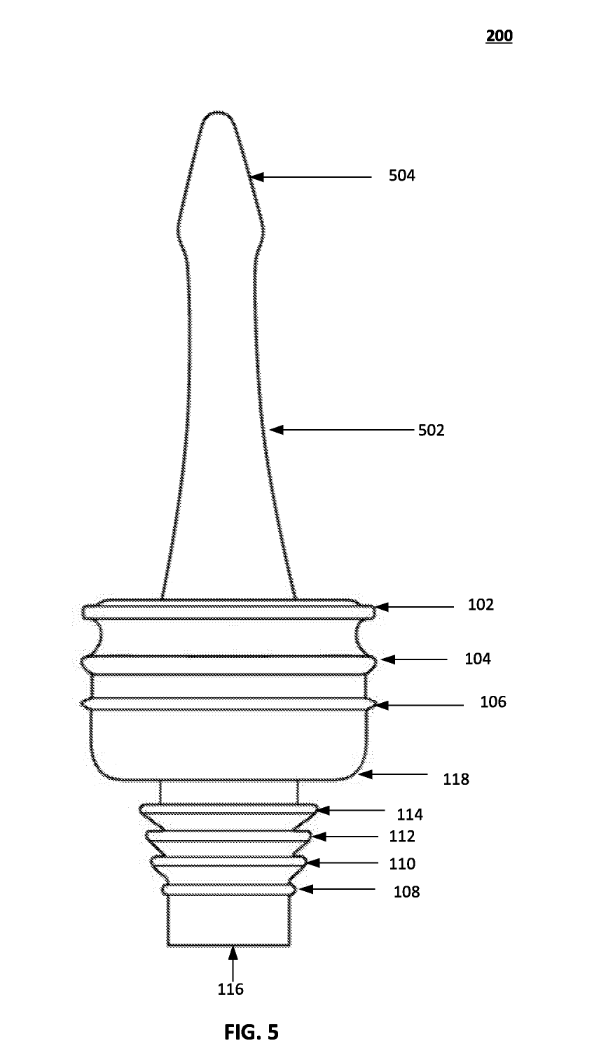

FIG. 5 illustrates an exemplary embodiment of enema device 200 from a view when the outside portion of attachment mechanism 100 is inverted. Numbers in FIG. 5 correspond to like numbers in FIGS. 1-4.

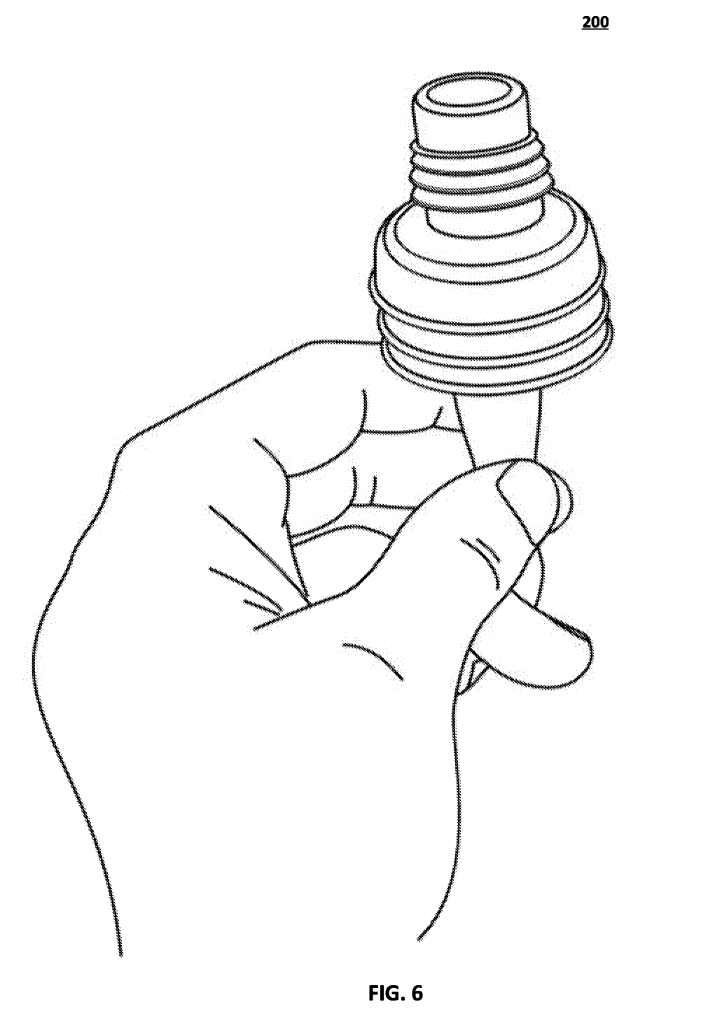

FIG. 6 illustrates an exemplary embodiment of enema device 200 in a person's hand. FIG. 6 is approximately drawn to scale. One of the benefits of enema device 200 is its portability. In some embodiments, enema device 200 is a total of less than 5.5 inches in height (as displayed in FIG. 8), thus allowing it to comfortably fit into most pockets or bags. The portability of the enema device 200 is enhanced with the fact that is capable of attaching, via attaching mechanism 100 to a plurality of bottles (e.g. water bottles). Thus, allowing an enema device to be discretely carried and used.

Many modifications and other embodiments will come to mind to one skilled in the art which the embodiments discussed herein pertain having the benefit of the teachings presented in the foregoing descriptions and the associated drawings.

1. A portable attachment device comprising:
an inner portion comprising a fluid path for distributing a solution from a solution source, wherein the inner portion is configured to fit at least partially inside a mouth of the solution source;
an outer portion surrounding the inner portion, wherein the outer portion is configured to surround a neck finish of the solution source, wherein the outer portion creates a first seal with the solution source and the inner portion creates a second with the solution source.

2. The portable attachment device of claim 1, wherein the outer portion surrounds a neck support ring of the solution source.

3. The portable attachment device of claim 2, wherein the outer portion comprises a first groove that is configured to interlock with one or more grooves located in the neck support ring of the solution source.

4. The portable attachment device of claim 3, wherein the outer portion comprises a lip that is configured to attach under the neck support ring of the solution source.

5. The portable attachment device of claim 1, wherein the inner portion comprises one or more inside grooves.

6. The portable attachment device of claim 5, wherein the one or more inside grooves are configured to be completely inserted into the mouth of the solution source.

7. A portable reusable enema device comprising:
a silicon tube comprising a pyramid-shaped end and an attachment end separated by an elongated portion, wherein the pyramid-shaped end is configured to be fully inserted into a person's rectum, and the attachment end is configured to surround a neck finish of a solution source, wherein the attachment end is configured to create a first seal with the solution source; and
a fluid path through the silicon tube, for distributing solution from the solution source to the person's rectum.

8. The portable reusable enema device of claim 7, silicon tube is less than 5.5 inches in height.

9. The portable reusable enema device of claim 7, wherein the solution is water.

10. The portable reusable enema device of claim 7, wherein the solution is a saline solution.

11. The portable reusable enema device of claim 7, wherein the solution source is a water bottle.

12. The portable reusable enema device of claim 7, wherein the attachment end further comprises a gripper portion, wherein at least part of the gripper portion protrudes in an outward direction from the portable reusable enema device.

13. The portable reusable enema device of claim 12, wherein the gripper portion comprises raised text.

14. The portable reusable enema device of claim 13, wherein the gripper portion is less than 1/16 inches in depth.

15. The portable reusable enema device of claim 7, wherein the solution is dispensed through the fluid path by applying a force to the solution source.

16. The portable reusable enema device of claim 15, wherein the force is a compression of the solution source performed by human hands.

17. The portable reusable enema device of claim 7, wherein the attachment end is configured to a second seal with the person's rectum, wherein the first and second seal are different.
